(19) United States
(12) Patent Application Publication
(10) Pub. No.: US 2021/0317080 A1
(43) Pub. Date: Oct. 14, 2021

(54) Polythiol Composition

(71) Applicant: PPG Industries Ohio, Inc., Cleveland, OH (US)

(72) Inventors: Charles R. Hickenboth, Zelienople, PA (US); Vivek Badarinarayana, Pittsburgh, PA (US); Andrew Davic, Cranberry Township, PA (US); Daryl Mains, Pittsburgh, PA (US)

(21) Appl. No.: 17/269,773

(22) PCT Filed: Aug. 19, 2019
(86) PCT No.: PCT/US2019/047038

§ 371 Date: Feb. 19, 2021

Related U.S. Application Data
(60) Provisional application No. 62/719,725, filed on Aug. 20, 2018.

(51) Int. Cl. C07C 323/64; C08G 18/38; C08G 18/77; C07D 251/52; C07D 339/08

(52) U.S. Cl. CPC C07C 323/64; C08G 18/3876; C07D 339/08; C07D 251/52; C08G 18/775

(57) ABSTRACT

The present invention relates to a polythiol composition that includes (a) a polythiol compound (A) having at least two thiol groups; and (b) a nitrogen-containing compound (B) represented by the following Formula (I),

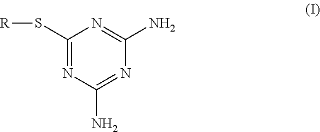

In Formula (I), —S—R is a residue of the polythiol compound (A). A peak area of the nitrogen-containing compound (B) is equal to or less than 3.0, with respect to a peak area of 100 of the polythiol compound (A). Each peak area for compounds (A) and (B) is determined by high performance liquid chromatography analysis. The present invention also relates to a polymerizable composition that includes the polythiol composition, and polymerizates thereof.

### CROSS-REFERENCE TO RELATED APPLICATION

This application is the United States national phase of International Application No. PCT/US2019/047038 filed Aug. 19, 2019 and claims priority to U.S. Provisional Application No. 62/719,725, filed Aug. 20, 2018, the disclosures of which are hereby incorporated by reference in their entirety.

### FIELD

The present invention relates to, a polythiol composition that includes a triazine compound that has bonded thereto a residue of a polythiol compound, in which the triazine compound is present, if at all, in an amount that is equal to or less than a threshold amount, polymerizable compositions that include the polythiol composition, and polymerizates thereof.

### BACKGROUND

Compared to glass lenses and transparencies, plastic lenses and transparencies are desirable in that they can provide reduced weight and improved physical properties, such as impact resistance. In the case of plastic lenses, such as ophthalmic lenses, to reduce the thickness of the lens and correspondingly optimize weight reduction, it is typically desirable that the plastic lens possess a combination of high refractive index and high Abbe number. A combination of high refractive index and high Abbe number can be obtained from polymerizable compositions that include a polythiol compound and a material that is reactive therewith, such as a polyiso(thio)cyanate.

In addition to high refractive index and high Abbe number, it is further desirable that a plastic lens also possess further optimized optical properties, such as reduced color (e.g., reduced yellowing), minimal haze, high transparency, minimal striations, minimal inclusions, minimal pre-release marks, and minimal edge marks. The synthesis of polythiol compounds used to prepare plastic lenses, can result in the formation of one or more contaminant compounds, which can result in reduced optical properties, such as increased color (e.g., increased yellowing), increased haze, reduced transparency, increased striations, increased inclusions, increased pre-release marks, and/or increased edge marks.

It would be desirable to identify the contaminant compound or compounds that are present in a polythiol composition, which can result in reduced optical properties of polymerizates prepared therefrom such as plastic lenses. It would be further desirable to develop and/or identify polythiol compositions, in which such contaminant compound(s) is/are present, if at all, in an amount that is equal to or less than a threshold amount.

### SUMMARY

In accordance with the present invention, there is provided a polythiol composition comprising: (a) a polythiol compound (A) comprising at least two thiol groups; and (b) a nitrogen-containing compound (B) represented by the following Formula (I),

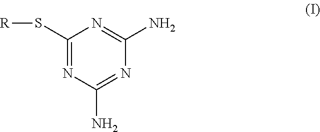

With reference to Formula (I), —S—R is a residue of the polythiol compound (A). A peak area of the nitrogen-containing compound (B) is equal to or less than 3.0, with respect to a peak area of 100 of the polythiol compound (A), wherein each peak area is determined by high performance liquid chromatography analysis.

### BRIEF DESCRIPTION OF THE DRAWING

FIG. 1 is a graphical representation of a high performance liquid chromatography (HPLC) chromatogram generated with regard to the analysis of 4-mercaptomethyl-1,8-dimercapto-3,6-dithiaoctane as discussed in Example 1 further herein.

### DETAILED DESCRIPTION

As used herein, the articles “a”, “an”, and “the” include plural referents unless otherwise expressly and unequivocally limited to one referent.

Unless otherwise indicated, all ranges or ratios disclosed herein are to be understood to encompass any and all subranges or subratios subsumed therein. For example, a stated range or ratio of “1 to 10” should be considered to include any and all subranges between (and inclusive of) the minimum value of 1 and the maximum value of 10; that is, all subranges or subratios beginning with a minimum value of 1 or more and ending with a maximum value of 10 or less, such as but not limited to, 1 to 6.1, 3.5 to 7.8, and 5.5 to 10.

Other than in the operating examples, or where otherwise indicated, all numbers expressing quantities of ingredients, reaction conditions, and so forth used in the specification and claims are to be understood as modified in all instances by the term “about”.

As used herein, the term “polyiso(thio)cyanate compound” and related terms means a compound that includes at least two iso(thio)cyanate groups selected from isothiocyanate group (—NCS), isocyanate group (—NCO), or combinations of isothiocyanate (—NCS) and isocyanate (—NCO) groups.

As used herein, recitations of “linear or branched” groups, such as linear or branched alkyl, are herein understood to include a methylene group or a methyl group; groups that are linear, such as linear C₂-C₁₀ alkyl groups; and groups that are appropriately branched, such as branched C₃-C₁₀ alkyl groups.

All documents, such as but not limited to issued patents and patent applications, referred to herein, and unless otherwise indicated, are to be considered to be “incorporated by reference” in their entirety.

By “threshold amount” of the nitrogen-containing compound (B) is meant a peak area of the nitrogen-containing compound (B) is equal to or less than 3.0, such as greater than 0 to less than or equal to 3.0 with respect to a peak area 100 of the polythiol compound (A), as determined by high performance liquid chromatography analysis.

The polythiol compositions of the present invention include a polythiol compound (A) that includes at least two thiol groups. With some embodiments, the polythiol compound (A) includes at least three thiol groups. With some further embodiments, the polythiol compound (A) includes from 2 to 10 thiol groups, or from 2 to 8 thiol groups, or from 2 to 6 thiol groups, or from 2 to 5 thiol groups, inclusive of the recited numbers. With some additional embodiments, the polythiol compound (A) includes 2, 3, 4, 5, or 6 thiol groups. The polythiol compound (A), with some embodiments, optionally includes at least one hydroxyl group, such as, but not limited to, 0, 1, 2, or 3 hydroxyl groups.

In accordance with some embodiments of the present invention, the polythiol compound (A) is represented by the following Formula (A-I):

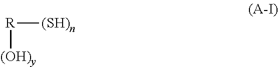

With reference to Formula (A-I), n is at least 2; y is equal to or greater than 0; and R is selected from linear or branched alkyl (such as at least divalent linear or branched alkyl), and cycloalkyl (such as at least divalent cycloalkyl), in each case optionally and independently including at least one sulfide linkage (—S—).

The linear or branched alkyl, and cycloalkyl, from which R of Formula (A-I) can be selected, in each case optionally and independently include at least one sulfide linkage (—S—), such as optionally 1 to 10 sulfide linkages, or optionally 1 to 7 sulfide linkages, or optionally 1 to 5 sulfide linkages, or optionally 1 to 4 sulfide linkages. With some embodiments, at least one carbon of, but less than all the carbons of, the linear or branched alkyl, and cycloalkyl is optionally replaced with at least one sulfide linkage (—S—). With some embodiments, the polythiol compound (A), such as represented by Formula (A-I), is free of one or more polysulfide linkages, —(S)t-, where t is at least 2; a thiol group (—SH) bonded directly to a sulfide linkage (—S—); and a hydroxyl group (—OH) bonded directly to a sulfide linkage (—S—). With some further embodiments, the linear or branched alkyl, and cycloalkyl, from which R of Formula (A-I) can be selected, are each free of: one or more polysulfide linkages, —(S)t-, where t is at least 2; a thiol group (—SH) bonded directly to a sulfide linkage (—S—); and a hydroxyl group (—OH) bonded directly to a sulfide linkage (—S—).

With further reference to Formula (A-I), and in accordance with some embodiments, subscript n is from 2 to 10, or from 2 to 8, or from 2 to 6, or from 2 to 5, inclusive of the recited values. With some additional embodiments, subscript n of Formula (A-I) is 2, 3, 4, 5, or 6. With additional reference to Formula (A-I), and in accordance with some embodiments, subscript y is 0, 1, 2, or 3.

With further reference to Formula (A-I), and in accordance with some embodiments of the present invention, n is from 2 to 6; y is from 0 to 3; and R is selected from at least divalent linear or branched C₁-C₂₀ alkyl (such as at least divalent linear or branched C₁-C₁₀ alkyl) and at least divalent C₃-C₁₂ cycloalkyl (such as at least divalent C₄-C₁₀ cycloalkyl), in each case optionally and independently including at least one sulfide linkage (—S—).

Examples of at least divalent linear or branched alkyl groups from which R of Formula (A-I) can be selected include, but are not limited to, methyl, ethyl, propyl, isopropyl, butyl, isobutyl, sec-butyl, tert-butyl, pentyl, neopentyl, hexyl, heptyl, octyl, nonyl and decyl, which are in each case at least divalent. Examples of at least divalent cycloalkyl groups from which R of Formula (A-I) can be selected include, but are not limited to, cyclopropyl, cyclobutyl, cyclopentyl, cyclohexyl, cycloheptyl, and cyclooctyl, which are in each case at least divalent.

The polythiol compound (A) is, with some embodiments, represented by the following Formula (A-II),

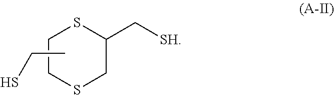

The polythiol compound (A) is, with some further embodiments, represented by the following Formula (A-III),

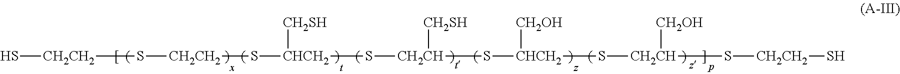

With reference to Formula (A-III), p is 0 to 4; and x, t, t′, z, and z′ are each independently 0 to 4 for each p. With some embodiments, and with further reference to Formula (A-III), p is 0 to 3; and x, t, t′, z, and z′ are each independently 0 to 3 for each p.

Examples of polythiols from which polythiol compound (A) can be selected, include, but are not limited to, 1,2-ethanedithiol, 1,2-propanedithiol, 1,3-propanedithiol, 1,2,3-propanetrithiol, tetrakis(mercaptomethyl)methane, trimethylolpropane tris(2-mercaptoacetate), trimethylolpropane tris(3-mercaptopropionate), trimethylolethane tris(2-mercaptoacetate), trimethylolethane tris(3-mercaptopropionate), pentaerythritol tetrakis(2-mercaptoacetate), pentaerythritol tetrakis(3-mercaptopropionate), 1,2,3-tris(mercaptomethylthio)propane, 1,2,3-tris(2-mercaptoethylthio)propane, 1,2,3-tris(3-mercaptopropylthio)propane, 1,5-dimercapto-3-thiapentane, 4-mercaptomethyl-1,8-dimercapto-3,6-dithiaoctane, 5,7-dimercaptomethyl-1,11-dimercapto-3,6,9-trithiaundecane, 4,7-dimercaptomethyl-1,11-dimercapto-3,6,9-trithiaundecane, 4,8-dimercaptomethyl-1,11-dimercapto-3,6,9-trithiaundecane, 7-(mercaptomethyl)-3,6,9,12-tetrathiatetradecane-1,14-dithiol, tetrakis(mercaptomethylthiomethyl)methane, tetrakis(2-mercaptoethylthiomethyl)methane, tetrakis (3-mercaptopropylthiomethyl)methane, bis(2,3-dimercaptopropyl)sulfide, 1,1,3,3-tetrakis(mercaptomethylthio)propane, 1,1,2,2-tetrakis(mercaptomethylthio)ethane, 4,6-bis(mercaptomethylthio)-1,3-dithiacyclohexane, tris(mercaptomethylthio)methane, tris(mercaptoethylthio)methane, and combinations of two or more thereof.

The polythiol compound (A) is, with some embodiments, selected from at least one of the following polythiol compounds represented by Formulas (A-1) through (A-5):

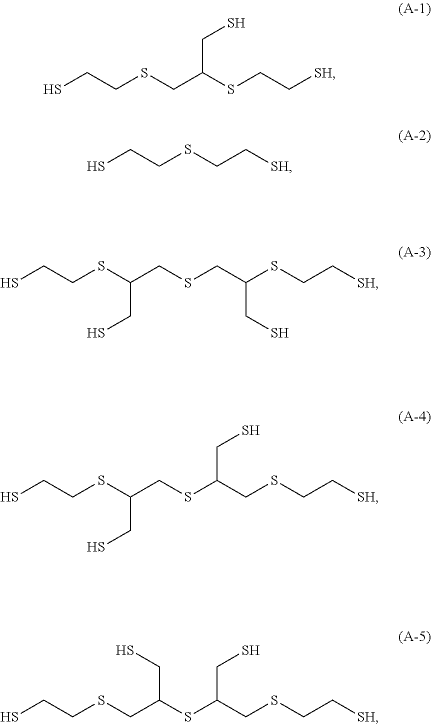 [SMILES: SCCSCC(CS)SCCS ; SCCSCCS ; SCCSC(CS)CSCC(CS)SCCS ; SCCSCC(CS)SCC(CS)SCCS ; SCCSCC(CS)SC(CS)CSCCS]

and combinations of two or more thereof.

Examples of polythiol compounds (A) that include a hydroxyl group include, but are not limited to, 2,3-dimercapto-1-propanol; 1,3-dimercaptopropan-2-ol; 2,3-bis((2-mercaptoethyl)thio)propan-1-ol; 1,3-bis((2-mercaptoethyl)thio)propan-2-ol; 3-mercapto-2-((2-mercaptoethyl)thio)propan-1-ol; 2-((2-mercaptoethyl)thio)-3-((2-((2-mercaptoethyl)thio)ethyl)thio)propan-1-ol; 2,3-bis((2-((2-mercaptoethyl)thio)ethyl)thio)propan-1-ol; glycerin bis(2-mercaptoacetate); glycerin bis (3-mercaptopropionate); 1,3-dimercapto-2-propanol; trimethylolpropane bis(2-mercapto acetate); trimethylolpropane bis (3-mercaptopropionate); pentaerythritol bis(2-mercaptoacetate); pentaerythritol tris(2-mercaptoacetate); pentaerythritol bis(3-mercaptopropionate); and pentaerythritol tris(3-mercaptopropionate).

The nitrogen-containing compound (B) includes at least one thiol group and optionally at least one hydroxyl group, with some embodiments. More particularly, the residue of the polythiol compound (A), —S—R, of the nitrogen-containing compound (B), includes at least one thiol group and optionally at least one hydroxyl group, with some embodiments.

The nitrogen-containing compound (B) of the polythiol compositions of the present invention is, with some embodiments, represented by Formula (I), in which —S—R is a residue of the polythiol compound (A). The —R group of the residue of the polythiol compound, —S—R, is as described previously herein with regard to the polythiol compound (A), such as being selected from linear or branched alkyl, and cycloalkyl, in each case optionally and independently including at least one sulfide linkage (—S—). With some embodiments, the residue of the polythiol compound (A), —S—R, of the nitrogen-containing compound (B), is free of (and does not have bonded thereto) a further nitrogen-containing compound (B) represented by Formula (I).

With some embodiments of the present invention, the nitrogen-containing compound (B) is represented by the following Formula (I-a):

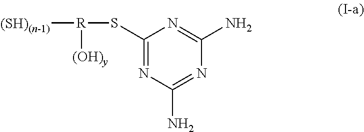

With reference to Formula (I-a), —S—R((SH)₍ₙ₋₁₎(OH)y is a residue of the polythiol compound (A), in which R, n, and y are each as described previously herein with regard to the polythiol compound (A). For purposes of non-limiting illustration, and with further reference to Formula (I-a), n is at least 2; y is equal to or greater than 0; and R is selected from linear or branched alkyl, or cycloalkyl, in each case optionally and independently including at least one sulfide linkage (—S—). With additional reference to Formula (I-a), and in accordance with some embodiments, n is from 2 to 6; y is from 0 to 3; and R is selected from linear or branched C₁-C₂₀ alkyl (such as linear or branched C₁-C₁₀ alkyl) and C₃-C₁₂ cycloalkyl (such as C₄-C₁₀ cycloalkyl), in each case optionally and independently including at least one sulfide linkage (—S—).

The nitrogen-containing compound (B), of the polythiol composition, includes at least two structural isomers, with some embodiments of the present invention. The structural isomers, with some embodiments, result from non-equivalent thiol groups (—SH) of the polythiol compound (A) covalently bonding with the triazine portion of the nitrogen-containing compound (B). For purposes of non-limiting illustration, when —S—R of the nitrogen-containing compound (B) represented by Formula (I) is a residue of the polythiol compound represented by Formula (A-1), the nitrogen-containing compound (B) can, with some embodiments, include one or more of three structural isomers represented by the following Formulas (B-1a), (B-1b), and (B-1c):

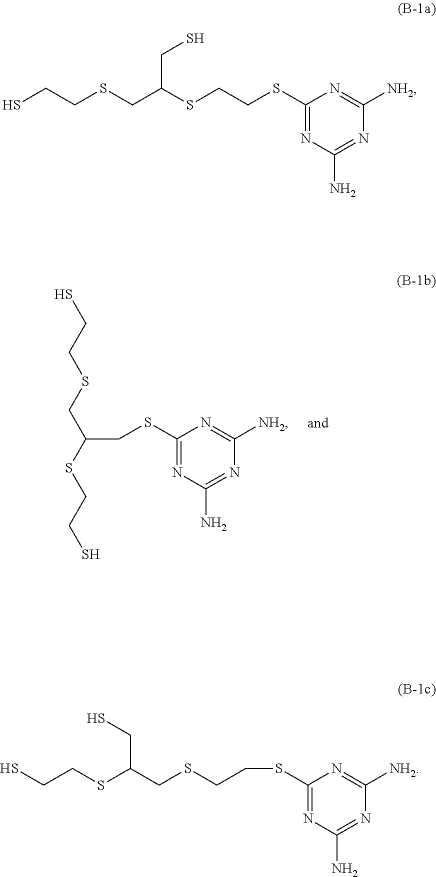 [SMILES: Nc1nc(N)nc(SCCSC(CS)CSCCS)n1 ; Nc1nc(N)nc(SCC(CSCCS)SCCS)n1 ; Nc1nc(N)nc(SCCSCC(CS)SCCS)n1]

The polythiol compound (A) is prepared, with some embodiments, by art-recognized synthetic methods that involve thiourea as a reactant. While not intending to be bound by any theory, it is believed, based on the facts presently at hand, that the nitrogen-containing compound (B) results from one or more side reactions (or co-reactions) involving thiourea.

The nitrogen-containing compound (B), of the polythiol composition of the present invention, is present, if at all, in an amount that is equal to or less than a threshold amount, with some embodiments of the present invention. The amount of nitrogen-containing compound (B) present within the polythiol composition is determined, with some embodiments, by high performance liquid chromatography (HPLC) analysis. The HPLC analysis is, with some further embodiments, coupled with high resolution mass spectrometry, such as high resolution Fourier transform mass spectroscopy, or high resolution Fourier transform mass spectroscopy with electrospray ion source.

A peak area of the nitrogen-containing compound (B) is equal to or less than 3.0, or equal to or less than 2.0, or equal to or less than 1.5, or equal to or less than 1.0, in each case with respect to a peak area of 100 of the polythiol compound (A), in which each peak area is determined by HPLC analysis, with some embodiments.

In accordance with some embodiments of the present invention, the conditions under which the HPLC analysis is conducted, for purposes of determining the relative peak areas of the nitrogen-containing compound (B) and the polythiol compound (A), include the following:
Column: YMC Pack ODS-A, 120 Å, 5 150×6 mm;
Mobile phase: acetonitrile/0.01 mol-potassium dihydrogen phosphate aqueous solution=60/40 (vol/vol);
Column temperature: 40° C.;
Flow rate: 1.0 ml/min;
Detector: UV detector, wavelength 230 nm;
Preparation of measurement solution: 160 mg of a sample dissolved and mixed in 10 ml of acetonitrile; and
Injection volume: 2 μl.

For further analysis of particular peaks (such as peaks at 4.6-5.4 min as described further herein in the Examples), the HPLC analysis is supplemented (or coupled), with some embodiments, with a suitable Fourier Transform Mass Spectrometry Elesctrospray ion source instrument, such as a Q-Exactive™ Hybrid Quadrupole-Orbitrap™ high resolution Fourier Transform Mass Spectrometry (FTMS) Electrospray ion source (ESI) (HR-FTMS-ESI), set at 140,000 mass resolution, commercially available from ThermoFisher Scientific. The HR-FTMS-ESI analysis is, with some embodiments, conducted as follows, in which the order of elution of peaks is retained and the retention times are similar, relative to the above described HPLC method:
Column: YMC Pack ODS-A, 120 Å, 3 100×3 mm;
Mobile phase: A=0.02% aqueous formic acid, B=acetonitrile, gradient according to the following Table 1;

TABLE 1

| Time | % A (0.02% formic | % B |
|---|---|---|
| (min) | acid in water) | (acetonitrile) |
| 0 | 50 | 50 |
| 8 | 40 | 60 |
| 13 | 2 | 98 |
| 25 | 2 | 98 |

Column temperature: 40° C.;
Flow rate: 0.3 ml/min;
Detector: UV detector, wavelength 230 and 250 nm;
Preparation of measurement solution: 160 mg of a sample dissolved and mixed in 10 ml of acetonitrile; and
Injection volume: 2.5 μl.

Using the above-described HR-FTMS-ESI analysis, the nitrogen-containing compound (0-2) (described in the Examples further herein) elutes at a retention time of 3.6-4.0 min.

With some embodiments, the HR-FTMS-ESI analysis is modified by replacing the ESI probe with an atmospheric solids analysis probe (ASAP), in which case the analysis is referred to herein as an HR-FTMS-ASAP analysis. The ASAP modification, with some embodiments, allows solids to be analyzed for molecular ions without further separation and with a reasonably high degree of sensitivity.

With some embodiments, a semi-preparatory HPLC method is used for purposes of isolating a larger amount of material for further analysis. Such semi-preparatory methods are described in further detail in the Examples herein.

The art-recognized methods by which the polythiol compound (A) is prepared can optionally result, with some embodiments, in the formation and presence of additional impurities. Such art-recognized synthetic methods include reactants such as, but not limited to, epihalohydrins, thiols, hydroxyls, thiourea, and halogen acids, and typically involve art-recognized work-up steps for isolating the resulting polythiol compound (A). Examples of such additional impurities include, but are not limited to, nitrogen containing impurities, sulfur containing impurities, halogen containing impurities, carbon containing impurities, and oxygen containing impurities. Further examples of such additional impurities include, but are not limited to, water; imines; ureas; amides; derivatives of melam and melem; condensation products of (thio)urea; condensation products of epihalohydrin; condensation products of alcohols, thiols, and mixtures thereof; alkanes optionally including one or more functional groups, such as, but not limited to, active hydrogen groups; (thio)ethers; and aromatic compounds containing one or more of carbonyls, alcohols, ethers, aldehydes, ketones, halogens, carboxylic acids, esters, amines, acyl halides, alkenes and alkynes. Such additional impurities can, with some embodiments, include oxidative products, which can result from the presence of oxygen and/or other oxidizing reagents present during synthesis. The additional impurities can, with further embodiments, include one or more metals, such as, but not limited to, lithium, sodium, potassium, cesium, magnesium, calcium, barium, aluminum, silicon, tin, lead, titanium, vanadium, chromium, manganese, cobalt, iron, nickel, copper, zinc, zirconium, palladium, silver, and platinum. The presence of metal impurities can result from the use of metal-containing reaction components, such as metal-containing reactants, metal-containing solvents, and metal-containing catalysts; and/or contact of reaction components with metal surfaces, such as metal piping and/or metal-lined reaction vessels. One or more of these additional impurities can, with some embodiments, be covalently bonded to the polythiol compound. The additional impurities can have a range of molecular weights, such as from 17 to 10,000, as determined by mass spectrometry. The additional impurities can be present in trace amounts, such as less than or equal to 1 part per million, up to 5 percent by weight, based on the total weight of the polythiol compound. The presence of such additional impurities can negatively affect the quality of the polythiol compound, compositions containing the polythiol compound, and/or molded articles prepared therefrom. As such, it is typically desirable to minimize the presence of such additional impurities.

The art-recognized methods by which the polythiol compound (A) is prepared can, with some embodiments, involve the use of acids and/or bases during isolation of the polythiol compound (A). As such, the polythiol compound (A) can, with some embodiments, have an acidic pH or a basic pH. For purposes of non-limiting illustration, if excess acid, such as HCl, is used, the isolated polythiol compound (A) can have an acidic pH. For purposes of further non-limiting illustration, if excess base, such as NaOH, is sued, the resulting polythiol compound (A) can have a basic pH.

In accordance with the present invention, there is also provided a polymerizable composition that includes (i) the polythiol composition of the present invention as described previously herein, which includes the polythiol compound (A), and the nitrogen-containing compound (B), which is present, if at all, in an amount that is equal to or less than a threshold amount; and (ii) a polyiso(thio)cyanate compound. The polyiso(thio)cyanate compound includes at least two iso(thio)cyanate groups. With some embodiments, the polyiso(thio)cyanate compound includes 2 to 6, or 2 to 5, or 2 to 4, or 2 or 3 iso(thio)cyanate groups.

Classes of polyiso(thio)cyanate compounds that can be used in the polymerizable composition of the present invention, include, but are not limited to, aliphatic polyiso(thio)cyanate compounds, such as linear or branched alkyl polyiso(thio)cyanate compounds and cycloalkyl polyiso(thio)cyanate compounds; aromatic polyiso(thio)cyanate compounds; polyiso(thio)cyanate compounds that include one or more sulfide linkages, such as aliphatic or aromatic polyiso(thio)cyanate compounds that include one or more sulfide linkages; polyiso(thio)cyanate compounds that include one or more disulfide linkages, such as aliphatic or aromatic polyiso(thio)cyanate compounds that include one or more disulfide linkages; and polyiso(thio)cyanate compounds that include both at least one isothiocyanate group and at least one isocyanate group.

Examples of linear or branched alkyl polyisocyanate compounds from which the polyiso(thio)cyanate compound can be selected, include, but are not limited to, hexamethylene diisocyanate, 1,5-pentane diisocyanate, 2,2-dimethylpentane diisocyanate, 2,2,4-trimethylhexane diisocyanate, butane diisocyanate, 2,4,4-trimethylhexamethylene diisocyanate, 1,6,11-undecane triisocyanate, 1,3,6-hexamethylene triisocyanate, 1,8-diisocyanato-4-isocyanatomethyloctane, bis(isocyanatoethyl)carbonate, bis(isocyanatoethyl)ether, lysine diisocyanatomethyl ester, lysine triisocyanate, and combinations of two or more thereof.

Examples of linear or branched alkyl polyisothiocyanate compounds from which the polyiso(thio)cyanate compound can be selected, include, but are not limited to, 1,2-diisothiocyanatoethane, 1,6-diisothiocyanatohexane, and combinations thereof.

Examples of cycloalkyl polyisocyanate compounds from which the polyiso(thio)cyanate compound can be selected, include, but are not limited to, isophorone diisocyanate, bis(isocyanatomethyl)cyclohexane, dicyclohexylmethane diisocyanate, cyclohexane diisocyanate, methylcyclohexane diisocyanate, dicyclohexyldimethylmethane isocyanate, 2,5-bis(isocyanatomethyl)-bicyclo[2.2.1]heptane, 2,6-bis(isocyanatomethyl)-bicyclo[2.2.1]heptane, 3,8-bis(isocyanatomethyl)tricyclodecane, 3,9-bis(isocyanatomethyl)tricyclodecane, 4,8-bis(isocyanatomethyl)tricyclodecane, 4,9-bis(isocyanatomethyl)tricyclodecane, bis(4-isocyanatocyclohexyl)methane, 1,3-bis(isocyanatomethyl)cyclohexane, 1,4-bis(isocyanatomethyl)cyclohexane, and combinations of two or more thereof.

A non-limiting example of a cycloalkyl polyisothiocyanate compound from which the polyiso(thio)cyanate compound can be selected, is cyclohexane diisothiocyanate.

Examples of aromatic polyisocyanate compounds from which the polyiso(thio)cyanate compound can be selected, include, but are not limited to, 1,2-diisocyanatobenzene, 1,3-diisocyanatobenzene, 1,4-diisocyanatobenzene, tolylene diisocyanate, 2,4-diisocyanatotoluene, 2,6-diisocyanatotoluene, ethylphenylene diisocyanate, isopropylphenylene diisocyanate, dimethylphenylene diisocyanate, diethylphenylene diisocyanate, diisopropylphenylene diisocyanate, trimethylbenzene triisocyanate, benzene triisocyanate, biphenyl diisocyanate, toluidine diisocyanate, 4,4′-methylenebis(phenyl isocyanate), 4,4′-methylenebis(2-methylphenyl isocyanate), bibenzyl-4,4′-diisocyanate, bis(isocyanatophenyl)ethylene, bis(isocyanatemethyl)benzene, m-xylylene diisocyanate, bis(isocyanatoethyl)benzene, bis(isocyanatopropyl)benzene, α,α,α′,α′-tetramethylxylylene diisocyanate, bis(isocyanatobutyl)benzene, bis(isocyanatomethyl)naphthalene, bis(isocyanatomethylphenyl)ether, bis(isocyanatoethyl)phthalate, 2,5-di(isocyanatomethyl)furan, and combinations of two or more thereof.

Examples of aromatic polyisothiocyanate compounds, from which the polyiso(thio)cyanate compound can be selected, include, but are not limited to, 1,2-diisothiocyanato benzene, 1,3-diisothiocyanato benzene, 1,4-diisothiocyanato benzene, 2,4-diisothiocyanato toluene, 2,5-diisothiocyanato-m-xylene, 4,4 ‘-methylenebis(phenyl isothiocyanate), 4,4’-methylenebis(2-methylphenyl isothiocyanate), 4,4-methylenebis (3-methylphenyl isothiocyanate), 4,4′-diisothiocyanato benzophenone, 4,4′-diisothiocyanato-3,3′-dimethyl benzophenone, or bis(4-isothiocyanatophenyl)ether, and combinations of two or more thereof.

Examples of aliphatic polyisocyanate compounds, such as linear or branched alkyl polyisocyanate compounds, that include one or more sulfide linkages, from which the polyiso(thio)cyanate compound can be selected, include, but are not limited to, bis(isocyanatomethyl)sulfide, bis(isocyanatoethyl)sulfide, bis(isocyanatopropyl)sulfide, bis(isocyanatohexyl)sulfide, bis(isocyanatomethyl)sulfone, bis(isocyanatomethyl)disulfide, bis(isocyanatoethyl)disulfide, bis(isocyanatopropyl)disulfide, bis(isocyanatomethylthio)methane, bis(isocyanatoethylthio)methane, bis(isocyanatomethylthio)ethane, bis(isocyanatoethylthio)ethane, 1,5-diisocyanato-2-isocyanatomethyl-3-thiapentane, 1,2,3-tris(isocyanatomethylthio)propane, 1,2,3-tris(isocyanatoethylthio)propane, 3,5-dithia-1,2,6,7-heptanetetra isocyanate, 2,6-diisocyanatomethyl-3,5-dithia-1,7-heptane diisocyanate, 2,5-diisocyanate methyl thiophene, 4-isocyanatoethylthio-2,6-dithia-1,8-octane diisocyanate, and combinations of two or more thereof.

Examples of aliphatic polyisothiocyanate compounds, such as linear or branched alkyl polyisothiocyanate compounds, that include one or more sulfide linkages, from which the polyiso(thio)cyanate compound can be selected, include, but are not limited to, bis(isocyanatomethyl)sulfide, bis(isocyanatoethyl)sulfide, bis(isocyanatopropyl)sulfide, bis(isocyanatohexyl)sulfide, bis(isocyanatomethyl)sulfone, bis(isocyanatomethyl)disulfide, bis(isocyanatoethyl)disulfide, bis(isocyanatopropyl)disulfide, bis(isocyanatomethylthio)methane, bis(isocyanatoethylthio)methane, bis(isocyanatomethylthio)ethane, bis(isocyanatoethylthio)ethane, 1,5-diisocyanato-2-isocyanatomethyl-3-thiapentane, 1,2,3-tris(isocyanatomethylthio)propane, 1,2,3-tris(isocyanatoethylthio)propane, 3,5-dithia-1,2,6,7-heptanetetra isocyanate, 2,6-diisocyanatomethyl-3,5-dithia-1,7-heptane diisocyanate, 2,5-diisocyanate methyl thiophene, 4-isocyanatoethylthio-2,6-dithia-1,8-octane diisocyanate, and combinations of two or more thereof.

Examples of cycloalkyl polyisothiocyanate compounds that include one or more sulfide linkages, from which the polyiso(thio)cyanate compound can be selected, include, but are not limited to, 2,5-diisothiocyanatothiophene, 2,5-diisothiocyanato-1,4-dithiane, and combinations thereof.

Examples of aromatic polyisocyanate compounds that include one or more sulfide linkages, from which the polyiso(thio)cyanate compound can be selected, include, but are not limited to, 2-isocyanatophenyl-4-isocyanatophenyl sulfide, bis(4-isocyanatophenyl)sulfide, bis(4-isocyanatomethylphenyl)sulfide, and combinations thereof.

Examples of aromatic polyisocyanate compounds that include one or more disulfide linkages, from which the polyiso(thio)cyanate compound can be selected, include, but are not limited to, bis(4-isocyanatophenyl)disulfide, bis(2-methyl-5-isocyanatophenyl)disulfide, bis(3-methyl-5-isocyanatophenyl)disulfide, bis (3-methyl-6-isocyanatophenyl)disulfide, bis(4-methyl-5-isocyanatophenyl)disulfide, bis(4-methoxy-3-isocyanatophenyl)disulfide, and combinations of two or more thereof.

Examples of polyisothiocyanate compounds that include at least one carbonyl-isothiocyanate group, from which the polyiso(thio)cyanate compound can be selected, include, but are not limited to, 1,3-benzene dicarbonyl diisothiocyanate, 1,4-benzene dicarbonyl diisothiocyanate, (2,2-pyridine)-4,4-dicarbonyl diisothiocyanate, and combinations of two or more thereof.

Examples of polyiso(thio)cyanate compounds that include at least one isocyanate group and at least one isothiocyanate group, from which the polyiso(thio)cyanate compound can be selected, include, but are not limited to, 1-isocyanato-6-isothiocyanatohexane, 1-isocyanato-4-isothiocyanatocyclohexane, 1-isocyanato-4-isothiocyanatobenzene, 4-methyl-3-isocyanato-1-isothiocyanatobenzene, 2-isocyanato-4,6-diisothiocyanate-1,3,5-triazine, 4-isocyanatophenyl-4-isothiocyanatophenyl sulfide, 2-isocyanatoethyl-2-isothiocyanatoethyl disulfide, and combinations of two or more thereof.

With some embodiments of the polymerizable composition of the present invention, the polyiso(thio)cyanate compound is a diisocyanate compound. The diisocyanate compound can be selected from those classes and examples of diisocyanate compounds recited previously herein, such as, but not limited to, hexamethylene diisocyanate, 2,2,4-trimethylhexane diisocyanate, isophorone diisocyanate, bis(isocyanatomethyl)cyclohexane, 4,4′-methylenebis(phenyl isocyanate), dicyclohexylmethane diisocyanate, 1,2-diisocyanatobenzene, 1,3-diisocyanatobenzene, 1,4-diisocyanatobenzene, tolylene diisocyanate, and combinations of two or more thereof.

The polythiol composition of the polymerizable composition, with some embodiments of the present invention, is present in an amount of from 15 to 85 percent by weight, or from 25 to 75 percent by weight, or from 45 to 55 percent by weight, the percent weights in each case being based on total weight of resin solids of the polymerizable composition.

The polyiso(thio)cyanate compound of the polymerizable composition, with some embodiments of the present invention, is present in an amount of from 15 to 85 percent by weight, or from 25 to 75 percent by weight, or from 45 to 55 percent by weight, the percent weights in each case being based on total weight of resin solids of the polymerizable composition.

With some embodiments of the polymerizable composition of the present invention, the molar ratio of iso(thio)cyanate groups of the polyiso(thio)cyanate compound to thiol groups of the polythiol composition can vary depending, for example, on the desired properties of the polymerizate prepared therefrom. The molar ratio of iso(thio)cyanate groups of the polyiso(thio)cyanate compound to thiol groups of the polythiol composition is, with some embodiments, from 0.5:1 to 10:1, or from 0.8:1 to 5:1, or from 0.9:1 to 4.5:1, or from 1:1 to 4:1.

The polymerizable composition of the present invention usually also includes one or more cure catalysts for catalyzing the reaction between the thiol groups of the polythiol composition and the iso(thio)cyanate groups of the polyiso(thio)cyanate compound. Classes of useful catalysts include, but are not limited to, metal compounds, such as, but not limited to, organic tin compounds, organic bismuth compounds, organic zinc compounds, organic zirconium compounds, organic aluminum compounds, organic nickel compounds, organic mercury compounds, and alkali metal compounds; and amine compounds, such as tertiary amine compounds, and quaternary ammonium compounds. Examples of organic tin compounds include, but are not limited to, tin(II) salts of carboxylic acids, such as tin(II) acetate, tin(II) octanoate, tin(II) ethylhexanoate and tin(II) laurate; tin(IV) compounds, such as dimethyltin dichloride, dibutyltin oxide, dibutyltin dichloride, dibutyltin diacetate, dibutyltin dilaurate, dibutyltin maleate and dioctyltin diacetate. Examples of suitable tertiary amine catalysts include, but are not limited to, diazabicyclo[2.2.2]octane and 1,5-diazabicyclo[4,3,0]non-5-ene. Examples of organic bismuth compounds include, but are not limited to, bismuth carboxylates. Examples of alkali metal compounds include, but are not limited to, alkali metal carboxylates, such as, but not limited to, potassium acetate, and potassium 2-ethylhexanoate. Examples of quaternary ammonium compounds include, but are not limited to, N-hydroxyalkyl quaternary ammonium carboxylates. With some embodiments, the catalyst is selected from tin(II) octanoate, dibutyltin(IV) dilaurate, and/or bismuth 2-ethylhexanoate. The catalyst is typically present in a catalytic amount, such as in an amount of 0.05 to 5.0 percent by weight, or 0.25 to 2.0 percent by weight, based on the total weight of resin solids in the polymerizable composition.

The polymerizable compositions of the present invention can, with some embodiments, optionally include one or more additives such as, but not limited to, waxes for flow and wetting; flow control agents, such as poly(2-ethylhexyl)acrylate; antioxidants; ultraviolet (UV) light absorbers; and/or tints, such as static dyes, dichroic dyes, photochromic compounds, and/or photochromic-dichroic compounds. Tints used with some embodiments of the polymerizable compositions of the present invention include, but are not limited to, one or more ANDARO® Tint Dispersion products, commercially available from PPG Industries, Inc. Examples of useful antioxidants and UV light absorbers include, but are not limited to, those available commercially from BASF under the trademarks IRGANOX and TINUVIN. These optional additives, when used, can be present in amounts up to 20 percent by weight, based on total solids weight of the polymerizable composition (excluding solvent).

The polymerizable compositions of the present can, with some embodiments, include one or more solvents, selected from water, organic solvents, and combinations thereof.

Classes of organic solvents that can be present in the polymerizable compositions of the present invention include, but are not limited to, alcohols, such as methanol, ethanol, n-propanol, iso-propanol, n-butanol, sec-butyl alcohol, tert-butyl alcohol, iso-butyl alcohol, furfuryl alcohol and tetrahydrofurfuryl alcohol; ketones or ketoalcohols, such as acetone, methyl ethyl ketone, and diacetone alcohol; ethers, such as dimethyl ether and methyl ethyl ether; cyclic ethers, such as tetrahydrofuran and dioxane; esters, such as ethyl acetate, ethyl lactate, ethylene carbonate and propylene carbonate; hydroxy functional ethers of alkylene glycols, such as butyl 2-hydroxyethyl ether, methyl 2-hydroxypropyl ether and phenyl 2-hydroxypropyl ether; nitrogen containing cyclic compounds, such as pyrrolidone, N-methyl-2-pyrrolidone and 1,3-dimethyl-2-imidazolidinone; sulfur containing compounds, such as dimethyl sulfoxide and tetramethylene sulfone; aromatic compounds, such as toluene, xylene, anisole, and butyl benzoate; and mixtures of aromatic compounds, such as, but not limited to, Aromatic 100 Fluid, which is a commercially available mixture of C₉-C₁₀ dialkyl- and trialkyl-benzenes.

Solvent(s) can be present in the polymerizable compositions of the present invention in an amount of from 5 to 95 percent by weight, or from 15 to 80 percent by weight, or from 30 to 60 percent by weight, in each case based on the total weight of the curable photochromic composition (including the weight of the solvent).

There is also provided, in accordance with the present invention, a polymerizate of the polymerizable composition of the present invention. The polymerizate is prepared by polymerizing (or curing) the polymerizable composition of the present invention.

The polymerizable composition of the present invention can be cured by any suitable method(s), so as to form the polymerizate of the present invention. With some further embodiments, the polymerizable composition is cured at ambient conditions, such as at room temperature of about 25° C. With some further embodiments, the polymerizable composition is cured by exposure to elevated temperature (in excess of ambient room temperature). As used herein, by “cured” is meant a three-dimensional crosslink network is formed by covalent bond formation, such as between the thiol groups of the polythiol composition and the iso(thio)cyanate groups of the polyiso(thio)cyanate compound. When cured at elevated temperature, the polymerizable composition can be referred to herein as a thermosetting polymerizable composition. The temperature at which the thermosetting polymerizable composition of the present invention is cured is variable and depends in part on the amount of time during which curing is conducted. With some embodiments, the polymerizable composition is cured at an elevated temperature of from 90° C. to 204° C., or from 100° C. to 177° C., or from 110° C. to 140° C., for a period of 20 to 240 minutes.

With some embodiments, the polymerizate of the present invention is selected from layers (including films and/or sheets), and 3-dimensional articles.

As used herein, the term “film” means a layer that is not self-supporting, such as, but not limited to, a coating. As used herein, the term “sheet” means a layer that is self-supporting.

Classes of 3-dimensional articles, that can be prepared from the polymerizable compositions of the present invention, and from which the polymerizate of the present invention can be selected, include, but are not limited to, optical elements, such as ophthalmic articles, display articles, camera lenses, windows, mirrors, active liquid crystal cell articles, and passive liquid crystal cell articles; and non-optical articles, such as, but not limited to, housings and support elements.

In accordance with the present invention there is also provided an optical element that includes a polymerizate of the polymerizable composition of the present invention. Classes of optical elements include, but are not limited to, ophthalmic articles, display articles, windows, mirrors, active liquid crystal cell articles, and passive liquid crystal cell articles.

Examples of ophthalmic articles include, but are not limited to, corrective lenses, non-corrective lenses, contact lenses, intra-ocular lenses, magnifying lenses, protective lenses, and visors. Examples of display article include, but are not limited to, screens, monitors, and security elements.

With some embodiments of the present invention, the optical element includes a substrate; and a layer over at least a portion of a surface of the substrate, in which the layer is a polymerizate of the polymerizable composition of the present invention. The substrate can include a matrix including an inorganic material, such as silica glass; an organic material, such as a thermoplastic polymer and/or a cured/polymerized polymer (such as a thermoset polymer); and combinations thereof. The layer of the optical element can be in the form of a film or sheet. The layer can be composed of a single layer or multiple layers that can be the same or different from each other. The layer of the optical element can be formed over the substrate by methods including, but not limited to, coating methods; lamination methods; and combinations thereof. Coating methods that can be used to form the layer of the optical element include, but are not limited to, spin coating; spray application; curtain coating; draw-down application methods; in-mold coating methods; and combinations thereof. Lamination methods that can be used to form the layer of the optical element include, but are not limited to, extrusion methods; adhesive lamination methods; in-mold lamination methods; and combinations thereof.

In accordance with the present invention, there is further provided a method of forming a molded article comprising (i) mixing together the polythiol composition of the present invention and a polyiso(thio)cyanate compound, thereby forming a polymerizable composition; (ii) introducing the polymerizable composition into a mold; and (iii) curing, at least partially, the polymerizable composition within said mold.

The polythiol composition of the present invention and the polyiso(thio)cyanate compound can be mixed together using any appropriate mixing method(s). Examples of suitable mixing methods include, but are not limited to, batch mixing, such as in an appropriate vessel, such as a mixing tank including one or more impellers; continuous mixing, such as in a static mixer and/or an extruder; impingement mixing, such as within a mixing chamber of a mold injection head; and combinations of such mixing methods.

After being formed by mixing, the polymerizable composition can be introduced into a mold by any appropriate method(s). The polymerizable composition can be introduced into a mold by methods including, but not limited to, pouring, such as from a beaker or other vessel; and/or injection, such as from a mold injector head. With some embodiments, the mold is a multiple-piece mold, such as a two-piece mold that includes at least one injection port and optionally one or more gaskets.

After introduction into the mold, the polymerizable composition of the present invention is cured, at least partially, within the mold. The term “cured” is as defined previously herein. The polymerizable composition can be cured at least partially within the mold at room temperature, such as about 25° C.; elevated temperature, such as from 90° C. to 204° C., or from 100° C. to 177° C., or from 110° C. to 140° C., for a period of 20 to 240 minutes; or any combination thereof. When cured partially within the mold, and in accordance with some embodiments, the polymerizable composition is cured at least to an extent that the resulting partially cured polymerizate can be removed intact from the mold. The partially cured polymerizate after removal from the mold is, with some embodiments, typically further cured. With some embodiments, the polymerizable composition is substantially completely cured within the mold.

With some embodiments, after curing, at least partially, the polymerizable composition of the present invention within the mold, the resulting polymerizate is typically removed from the mold. The polymerizate can be subjected to one or more additional steps such as, but not limited to, grinding; surface cleaning; surface treatment, such as etching and/or plasma treatments; forming one or more layers over at least one surface of the polymerizate, such as, but not limited to, protective layers, tinted layers, and/or anti-reflective layers; and combinations thereof.

In accordance with the present invention, there is additionally provided a nitrogen-containing compound (B) represented by Formula (I) as shown previously herein, in which —S—R is a residue of a polythiol compound (A) that comprises at least two thiol groups. The nitrogen-containing compound (B) and polythiol residue —S—R of the polythiol compound (A) are each as described previously herein. The nitrogen-containing compound can, with some embodiments, be isolated from a polythiol composition, such as the polythiol composition of the present invention, by art-recognized preparative HPLC methods. The nitrogen-containing compound can, with some further embodiments, be prepared synthetically, such as by reacting together a halo-triazine, such as 2-chloro-4,6-diamino-1,3,5-triazine, and a polythiol compound, followed by art-recognized work-up procedures.

The present invention is more particularly described in the following examples, which are intended to be illustrative only, since numerous modifications and variations therein will be apparent to those skilled in the art. Unless otherwise specified, all parts and all percentages are by weight.

### EXAMPLES

### Example 1

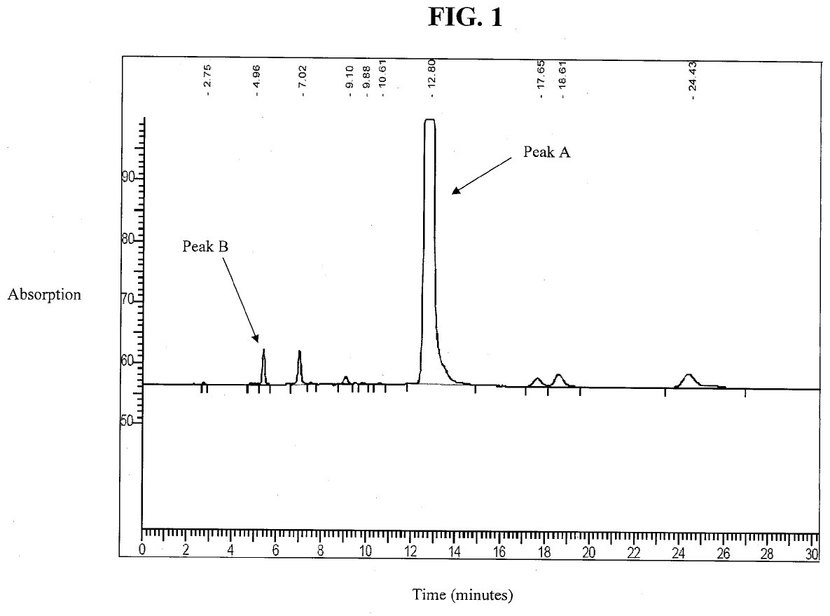

A sample of 4-mercaptomethyl-1,8-dimercapto-3,6-dithiaoctane was analyzed in accordance with the HPLC analytical method and conditions described previously in the specification herein. With reference to FIG. 1, the area of Peak B (observed at 4.6-5.4 min; and corresponding to a mixture including structural isomers of a nitrogen-containing compound B having 4-mercaptomethyl-1,8-dimercapto-3,6-dithiaoctane covalently bonded thereto) was found to be 0.1 relative to a peak area 100 of Peak A (corresponding to 4-mercaptomethyl-1,8-dimercapto-3,6-dithiaoctane). The components of Peak B were further analyzed in accordance with the HR-FTMS-ESI instrument and conditions described previously in the specification herein with a gradient mobile phase as shown in Table 1, and were found to be a mixture of a polythiol compound (0-1) represented by the following Formula (A-6) with a retention time of 4.3 to 4.5 minutes, and a mixture of nitrogen-containing compounds (0-2) represented by Formulas (B-1a), (B-1b), and (B-1c) as shown previously herein with a retention time of 3.6 to 4.0 minutes.

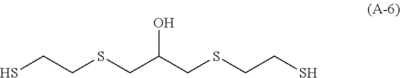 [SMILES: OC(CSCCS)CSCCS]

The HR-FTMS-ESI results for the polythiol compound (0-1) and the mixture of nitrogen-containing compounds (0-2) are summarized as follows:
(O-1): HR-FTMS-ESI calculated for C₉H₂₀NOS₄ (M+CH₃CN+H)⁺286.0422, found 286.0428; and
(O-2): HR-FTMS-ESI calculated for C₁₀H₂₀N₅S₅ (M+H)⁺ 370.0317, found 370.0319

The mixture of nitrogen-containing compounds (0-2) was isolated by semi-prep HPLC using a Waters Alliance 2695 Separations Module fitted with a Waters 996 Photoarray Detector at 230 nm, with a flow rate of 2 mL/min at 40° C. and a solvent gradient according to Table 2 below. The mixture of nitrogen-containing compounds (0-2) eluted at 6-8 minutes under these conditions. The isolated material was further characterized by ¹H-NMR and ¹³C-NMR, which confirmed the structures thereof as represented by Formulas (B-1a), (B-1b), and (B-1c). The ¹H-NMR and ¹³C-NMR results are summarized as follows:

¹H-NMR (500 MHz, D6-DMSO) δ 2.6-2.8 ppm (m, CH₂), 3.1-3.5 ppm (m, CH₂; CH); 6.8-7.0 ppm (br s, C—NH₂).

¹³C-NMR (500 MHz, D6-DMSO): δ ppm 178.6 (1C, s, CH₂SC(═N)—N), 166.1 (2C, s, N═C(NH₂)—N═C), 43-48 (s, CH; CH₂), 24-40 (s, CH₂)

TABLE 2

| Time | % A (0.02% formic | % B |
|---|---|---|
| (min) | acid in water) | (acetonitrile) |
| 0 | 50 | 50 |
| 7 | 40 | 60 |
| 13 | 2 | 98 |
| 25 | 2 | 98 |

The ¹³C-NMR results are consistent with the mixture of nitrogen-containing compounds (0-2) including a compound represented by Formula (B-1a) (depicted as one regioisomer), and the chemical shifts assigned to the triazine carbons agree reasonably well with the ¹³C-NMR chemical shifts assigned to the triazine carbons as reported for S-methyl-diamino-S-triazine (6-(methylthio)-1,3,5-triazine-2,4-diamine) by Murakami et al. (J. Pesticide Sci. 18, 1993, 147-154, reported 165.59 for and 178.86 ppm for carbons in triazine ring, in D6-DMSO).

### Example 2

A mixture of nitrogen-containing compounds (0-2) was prepared for purposes of confirming the structures as represented by Formulas (B-1a), (B-1b), and (B-1c). The mixture of nitrogen-containing compounds (0-2) was prepared via an independent route, by the reaction of 4-mercaptomethyl-1,8-dimercapto-3,6-dithiaoctane with chlorodiamino-s-triazine (6-chloro-1,3,5-trazine-2,4-diamine). A procedure similar to that reported by Muldoon et al. was used (J. Agric. Food Chem., 1994, 42, pp 747-755). Into a 250 mL single necked round-bottomed flask was added 100 mL of 95:5 ethanol:isopropyl alcohol and chlorodiamino-s-triazine (0.725 g, 5 mmol). To the resulting suspension was added a solution of 0.33 g of 85% KOH (5 mmol) and 1.3 g of 4-mercaptomethyl-1,8-dimercapto-3,6-dithiaoctane (5 mmol), both dissolved in 10 mL of 95:5 ethanol:isopropyl alcohol. This suspension was warmed to reflux for 24 hours, then cooled and filtered through coarse filter paper. Thin layer chromatography (TLC) analysis (5% methanol in methylene chloride) indicated that the starting triazine was largely consumed, and several new spots with Rf between 0.3 and 0.5 were produced. To the filtrate was added approximately 20 g of silica, then the solvent was removed under vacuum. The dried silica containing the crude reaction mixture was placed on a fitted funnel and washed with toluene (100 mL), methylene chloride (100 mL) and 20% methanol in toluene. TLC analysis indicated that the new products, along with residual 4-mercaptomethyl-1,8-dimercapto-3,6-dithiaoctane starting material, were contained in the first toluene fraction. The solvent was removed under vacuum to yield a white semi-solid material (approximately 1 g). Approximately 250 mg of the semi-solid material was purified by flash chromatography on silica gel, (100% methylene chloride →5% methanol in methylene chloride), yielding the mixture of nitrogen-containing compounds (0-2) as a light colored oil and a mixture of isomers.

Fourier Transform Infrared (FTIR) analysis of the isolated product was consistent with the mixture of nitrogen-containing compounds (0-2) having structures as represented by Formulas (B-1a), (B-1b), and (B-1c). In particular, the following key absorbances were observed: 1530 cm⁻¹ (triazine ring quadrant stretch); 1438 cm⁻¹ (triazine ring semi-circle stretch); and 808 cm⁻¹ (melamine ring sextant out of plane bending).

Murakami et al. (citation provided above) previously identified an IR absorption at approximately 1525 cm⁻¹ as an indicator of melamine ring stretching in 6-(methylthio)-1,3,5-triazine-2,4-diamine.

Furthermore, Welcher et al. (Journal of the American Chemical Society, 1959 (81), 5663) identified an IR absorption at approximately 810 cm⁻¹ as a key indicator of S-alkyl substituted triazine-2,4-diamine structures.

The FTIR analysis of the mixture of nitrogen-containing compounds (0-2) of this example is summarized as follows: 3321 cm⁻¹ (br, NH stretch), 3188 cm⁻¹ (br, NH stretch), 2910 cm⁻¹ (C—H stretch), 2540 cm⁻¹ (SH stretch), 1610 cm⁻¹ (C═N stretch), 1530 cm⁻¹ (Triazine ring quadrant stretch), 1438 cm⁻¹ (triazine ring semi-circle stretch), 808 cm⁻¹ (melamine ring sextant out of plane bending).

The mixture of nitrogen-containing compounds (0-2) of this example was further purified by semi-prep HPLC using the column described above and the eluent described in Table 3 below. The mixture of nitrogen-containing compounds (0-2) in an amount of 4 mg was isolated from 15 mg of sample in this manner. The mixture of nitrogen-containing compounds (0-2) eluted at 6-8 min under these conditions. The purity of the isolated material was estimated to be >90%. Analytical data (¹H-NMR; ¹³C-NMR; UV-VIS; and HR-FTMS-ESI) was consistent with the mixture of nitrogen-containing compounds (0-2) having structures represented by Formulas (B-1a), (B-1b), and (B-1c), and is summarized as follows:

¹H-NMR (500 MHz, D6-DMSO) δ 2.6-2.8 ppm (m, CH₂), 3.1-3.5 ppm (m, CH₂; CH); 6.8-7.0 ppm (br s, C—NH₂);

¹³C-NMR (500 MHz, D6-DMSO) δ ppm 178.6 (1C, s, CH₂SC(═N)—N), 166.1 (2C, s, N═C(NH₂)—N═C), 43-48 (s, CH; CH₂), 24-40 (s, CH₂); and

HR-FTMS-ESI calculated for C₁₀H₂₀N₅S₅(M+H)⁺370.0317, found 370.0316 UV-Vis λ max 206 nm.

TABLE 3

| | % A | % B |
|---|---|---|
| Time | (water) | (acetonitrile) |
| 0 | 50 | 50 |
| 8 | 40 | 60 |
| 13 | 2 | 98 |
| 25 | 2 | 98 |

The ¹H-NMR and ¹³C-NMR results were further confirmed with ¹³C¹H NMR-HSQC and ¹³C¹H NMR-HMBC analysis. In particular, correlation was observed between the ¹³C with a chemical shift at 178.6 ppm (CH₂SC(═N)—N in the triazine ring) and the ¹Hs with a chemical shift at 3.1-3.5 ppm (multiplets). These correlations suggest that the nitrogen-containing compound (0-2) comprises at least two different regioisomers and also have direct connectivity of the triazine moiety to aliphatic thioether chains. The ¹³C¹H NMR-HSQC and ¹³C¹H NMR-HMBC results are summarized as follows:

¹³C¹H NMR-HSQC (500 MHz, D6-DMSO) δ ppm 24-26 ppm (2.6-2.8 ppm, C—H₂), 28-40 ppm (2.6-3.4 ppm, C—H₂), 46-50 ppm (2.8-3.5 ppm, CH, CH₂); and

¹³C¹H NMR-HMBC (500 MHz, D6-DMSO) δ ppm 24-26 ppm (2.6-2.8 ppm, CH₂CH₂SH), 28-40 ppm (2.6-3.4 ppm, CH₂SCH₂CH₂), 46-50 ppm (2.8-3.4 ppm, (CH₂)₂CHS; CH₂SCH₂CH₂), 166 ppm (6.8-7.0 ppm, N═C(NH₂)—N═C), 179 (3.1-3.5 ppm; 6-8 ppm, CH₂SC(═N)—N═C—NH₂).

### Example 3

In the present Example 3, the polythiol compound (0-1) was isolated from a 300 mg sample of 4-mercaptomethyl-1,8-dimercapto-3,6-dithiaoctane by semi-prep HPLC as described previously in Example 1, with an eluent as described in Table 2. The polythiol (0-1) eluted at 6-8 min under these conditions. The purity of the isolated material (yield: 2 mg) was estimated to be >90%. Analytical data was consistent with the polythiol (0-1) having the structure represented by Formula (A-6), which is summarized as follows:

¹H-NMR (500 MHz, D6-DMSO) δ 2.5-2.8 ppm (12H, m, CH₂), 3.7 ppm (1H, pentet, H—COH); 5.1 ppm (1H, br s, C—OH);

¹³C-NMR (125 MHz, D6-DMSO)) δ ppm 24-26 (C—H₂), 36-39 (C—H₂), 70.9 (H—COH); and

HR-FTMS-ESI calculated for C₉H₂₀NOS₄ (M+CH₃CN+H)+286.0428, found 286.0417.

The structure of the polythiol (0-1), as represented by Formula (A-6), was further confirmed with ¹³C¹H NMR-HSQC and ¹³C¹H NMR-HMBC analysis. Correlations exist between the carbon next to the oxygen at 70.1 ppm chemical shift and the CH and CH₂ peaks in the SCH₂CH(OH)CH₂S group. These correlations suggest that the polythiol (0-1) has one primary regioisomer with possible smaller numbers of other regioisomers. No direct connectivity of triazine functionality to the molecule was observed. The ¹³C¹H NMR-HSQC and ¹³C¹H NMR-HMBC analysis results are summarized as follows:

¹³C¹H NMR-HSQC (500 MHz, D6-DMSO) δ ppm 24-26 ppm (2.6-2.8 ppm, C—H₂), 36-39 ppm (2.5-2.8 ppm, C—H₂), 70.9 ppm (3.7 ppm, H—COH); and

¹³C¹H NMR-HMBC (500 MHz, D6-DMSO) δ ppm 24-26 ppm (2.6-2.8 ppm, CH₂CH₂SH), 36-39 (2.5-2.8 ppm, CH₂CH₂CH₂; SCH₂CH), 70.9 ppm (3.7 ppm, H—COH).

### Example 4

The present Example 4 is directed to the isolation of the mixture of nitrogen-containing compounds (0-2) from a sample of 4-mercaptomethyl-1,8-dimercapto-3,6-dithiaoctane. For purposes of separating the polythiol compound (0-1) and the mixture of nitrogen-containing compounds (0-2), 2 mg of the mixture of nitrogen-containing compounds (0-2) was isolated from 100 mg of 4-mercaptomethyl-1,8-dimercapto-3,6-dithiaoctane by semi-prep HPLC as described previously in Example 1, using a solvent gradient according to Table 2 above. The mixture of nitrogen-containing compounds (0-2) eluted at 6-8 min under these conditions. The purity of the isolated material was estimated to be >90%. Analytical data was consistent with the mixture of nitrogen-containing compounds (0-2) including a compound having a structure as represented by Formula (B-1a), which is summarized as follows:

¹H NMR (500 MHz, D6-DMSO) δ 2.6-2.8 ppm (m, CH₂), 3.1-3.5 ppm (m, CH₂; CH); 6.8-7.0 ppm (br s, C—NH₂); and

HR-FTMS (ASAP) calculated for C₁₀H₂₀N₅S₅(M+H)⁺370.0317, found 370.0314.

The mixture of nitrogen-containing compounds (0-2) including a compound represented by Formula (B-1a), was further confirmed with ¹³C¹H NMR-HSQC and ¹³C¹H NMR-HMBC analysis. In particular, correlation was observed between the 178.6 ppm triazine ring and the 3.1-3.5 ppm multiplets. These correlations suggest that the mixture of nitrogen-containing compounds (0-2) includes at least two different regioisomers and direct connectivity of the triazine to 4-mercaptomethyl-1,8-dimercapto-3,6-dithiaoctane. The ¹³C¹H NMR-HSQC and ¹³C¹H NMR-HMBC analytical results are summarized as follows:

¹³C¹H NMR-HSQC (500 MHz, D6-DMSO) δ ppm 24-26 ppm (2.6-2.8 ppm, C—H₂), 28-39 ppm (2.6-3.4 ppm, C—H₂), 46-50 ppm (2.8-3.5 ppm, CH, CH₂); and

¹³C¹H NMR-HMBC (500 MHz, D6-DMSO) δ ppm 24-26 ppm (2.6-2.8 ppm, CH₂CH₂SH), 28-39 ppm (2.6-3.4 ppm, CH₂SCH₂CH₂), 46-50 ppm (2.8-3.4 ppm, (CH₂)₂CHS; CH₂SCH₂CH₂), 166 ppm (6.8-7.0 ppm, N═C(NH₂)—N═C), 179 (3.1-3.5 ppm; 6-8 ppm, CH₂SC(═N)—N═C—NH₂).

The present invention can be further characterized by one or more of the following non-limiting clauses:

Clause 1: A polythiol composition comprising:

(a) a polythiol compound (A) comprising at least two thiol groups; and

(b) a nitrogen-containing compound (B) represented by the following Formula (I),

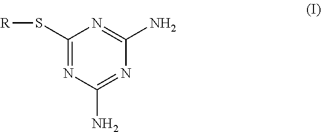

wherein with Formula (I), —S—R is a residue of the polythiol compound (A), and

wherein a peak area of the nitrogen-containing compound (B) is equal to or less than 3.0, with respect to a peak area of 100 of the polythiol compound (A), wherein each peak area is determined by high performance liquid chromatography analysis.

Clause 2: The polythiol composition of clause 1, wherein the polythiol compound (A) comprises at least three thiol groups.

Clause 3: The polythiol composition of clause 1 or 2, wherein the polythiol compound (A) is represented by the following Formula (A-I):

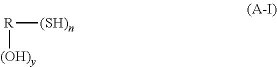

wherein n is at least 2,

y is equal to or greater than 0, and

R is selected from linear or branched alkyl, and cycloalkyl, in each case optionally and independently comprising at least one sulfide linkage.

Clause 4: The polythiol composition of clause 3, wherein for the polythiol compound (A) represented by Formula (A-I),

n is from 2 to 6,

y is from 0 to 3, and

R is selected from linear or branched C₁-C₁₀ alkyl, and C₄-C₁₀ cycloalkyl, in each case optionally and independently comprising at least one sulfide linkage.

Clause 5: The polythiol composition of clauses 1, 3 or 4, wherein the polythiol compound (A) is represented by the following Formula (A-II),

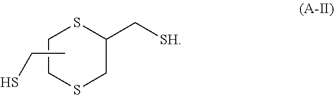

Clause 6: The polythiol composition of any one of clauses 1-4, wherein the polythiol compound (A) is represented by the following Formula (A-III),

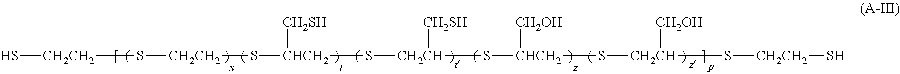

wherein p is 0 to 4, and

x, t, t′, z, and z′ are each independently 0 to 4 for each p.

Clause 7: The polythiol composition of clause 1 or 6, wherein the polythiol compound (A) is selected from at least one of the following polythiol compounds represented by Formulas (A-1) through (A-5):

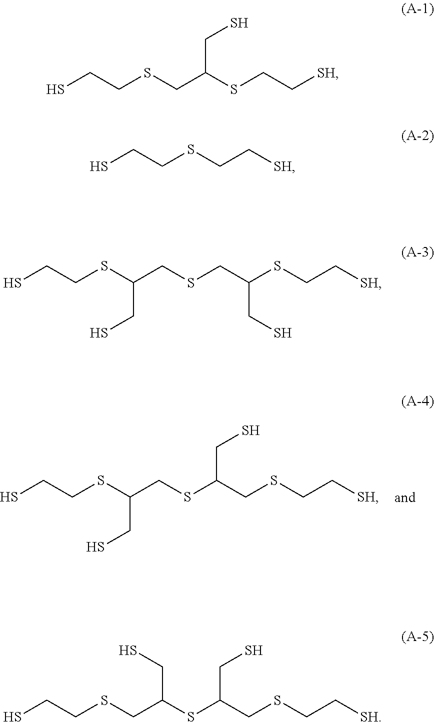 [SMILES: SCCSCC(CS)SCCS ; SCCSCCS ; SCCSC(CS)CSCC(CS)SCCS ; SCCSCC(CS)SCC(CS)SCCS ; SCCSCC(CS)SC(CS)CSCCS]

Clause 8: The polythiol composition of clauses 1, 2, 3, 4, or 6, wherein the nitrogen-containing compound (B) comprises at least one thiol group and optionally at least one hydroxyl group.

Clause 9: The polythiol composition of any one of clauses 1-8, wherein the nitrogen-containing compound (B) comprises at least two structural isomers.

Clause 10: A polymerizable composition comprising:

(i) the polythiol composition of any one of clauses 1-9; and

(ii) a polyiso(thio)cyanate compound.

Clause 11: The polymerizable composition of clause 10, wherein the polyiso(thio)cyanate compound is a diisocyanate compound.

Clause 12: A polymerizate of the polymerizable composition of clause 10.

Clause 13: An optical element comprising a polymerizate of the polymerizable composition of clause 10.

Clause 14: A method of forming a molded article comprising:

(i) mixing together the polythiol composition of any one of clauses 1-9 and a polyiso(thio)cyanate compound, thereby forming a polymerizable composition;

(ii) introducing said polymerizable composition into a mold; and

(iii) curing, at least partially, said polymerizable composition within said mold.

Clause 15: A nitrogen-containing compound (B) represented by the following Formula (I),

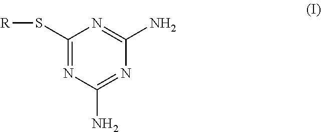

wherein with Formula (I), —S—R is a residue of a polythiol compound (A) comprising at least two thiol groups.

Clause 16: The polythiol composition of any one of clauses 1 to 9, wherein the peak area of the nitrogen-containing compound (B) is greater than 0 to less than or equal to 3.0 with respect to a peak area 100 of the polythiol compound (A), wherein each peak is determined by high performance liquid chromatography analysis.

Clause 17: The polythiol composition of clause 1 or 16, wherein the peak area of the nitrogen-containing compound (B) is equal to or less than 2.0, such as equal to or less than 1.5, such as equal to or less than 1.0, with respect to a peak area 100 of the polythiol compound (A), in which each peak area is determined by high performance liquid chromatography analysis.

The present invention has been described with reference to specific details of particular embodiments thereof. It is not intended that such details be regarded as limitations upon the scope of the invention except insofar as and to the extent that they are included in the accompanying claims.

1. A polythiol composition comprising:
(a) a polythiol compound (A) comprising at least two thiol groups; and
(b) a nitrogen-containing compound (B) represented by the following Formula 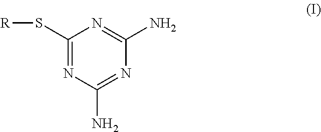
wherein with Formula (I), —S—R is a residue of said polythiol compound (A), and
wherein a peak area of said nitrogen-containing compound (B) is equal to or less than 3.0, with respect to a peak area of 100 of said polythiol compound (A), wherein each peak area is determined by high performance liquid chromatography analysis.

2. The polythiol composition of claim 1, wherein said polythiol compound (A) comprises at least three thiol groups.

3. The polythiol composition of claim 1, wherein said polythiol compound (A) is represented by the following Formula (A-I): 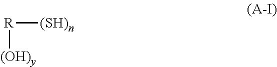
wherein n is at least 2,
y is equal to or greater than 0, and
R is selected from linear or branched alkyl, and cycloalkyl, in each case optionally and independently comprising at least one sulfide linkage.

4. The polythiol composition of claim 3, wherein for said polythiol compound (A) represented by Formula (A-I),
n is from 2 to 6,
y is from 0 to 3, and
R is selected from linear or branched C₁-C₁₀ alkyl, and C₄-C₁₀ cycloalkyl, in each case optionally and independently comprising at least one sulfide linkage.

5. The polythiol composition of claim 4, wherein said polythiol compound (A) is represented by the following Formula (A-II),

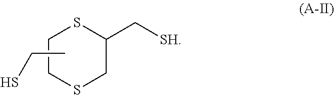

6. The polythiol composition of claim 4, wherein said polythiol compound (A) is represented by the following Formula (A-III), 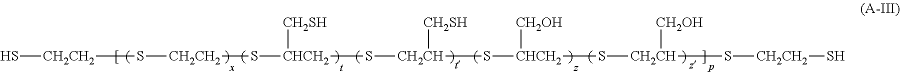
wherein p is 0 to 4, and
x, t, t′, z, and z′ are each independently 0 to 4 for each p.

7. The polythiol composition of claim 6, wherein said polythiol compound (A) is selected from at least one of the following,

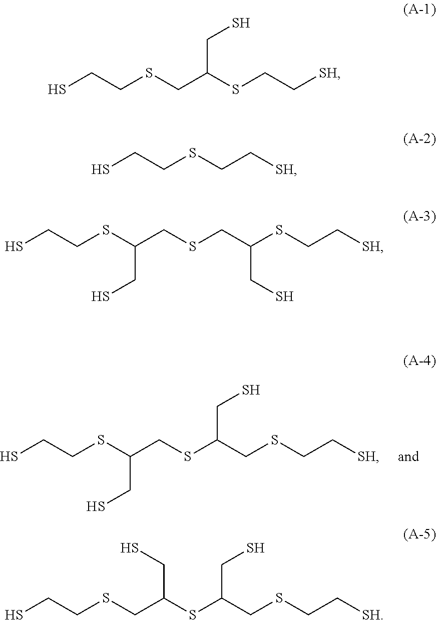 [SMILES: SCCSCC(CS)SCCS ; SCCSCCS ; SCCSC(CS)CSCC(CS)SCCS ; SCCSCC(CS)SCC(CS)SCCS ; SCCSCC(CS)SC(CS)CSCCS]

8. The polythiol composition of claim 1, wherein said nitrogen-containing compound (B) comprises at least one thiol group and optionally at least one hydroxyl group.

9. The polythiol composition of claim 1, wherein said nitrogen-containing compound (B) comprises at least two structural isomers.

10. A polymerizable composition comprising:
(i) said polythiol composition of claim 1; and
(ii) a polyiso(thio)cyanate compound.

11. The polymerizable composition of claim 10, wherein said polyiso(thio)cyanate compound is a diisocyanate compound.

12. A polymerizate of said polymerizable composition of claim 10.

13. An optical element comprising a polymerizate of said polymerizable composition of claim 10.

14. A method of forming a molded article comprising:
(i) mixing together said polythiol composition of claim 1 and a polyiso(thio)cyanate compound, thereby forming a polymerizable composition;
(ii) introducing said polymerizable composition into a mold; and
(iii) curing, at least partially, said polymerizable composition within said mold.

15. A nitrogen-containing compound (B) represented by the following Formula (I), 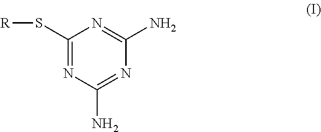
wherein with Formula (I), —S—R is a residue of a polythiol compound (A) comprising at least two thiol groups.
